(19) United States
(12) Patent Application Publication
(10) Pub. No.: US 2008/0194003 A1
(43) Pub. Date: Aug. 14, 2008

(54) System for producing food and feed

(76) Inventor: Jere Northrop, Amherst, NY (US)

Correspondence Address: JERE NORTHROP, 1961 TONAWANDA CREEK ROAD, AMHERST, NY, 14228, US

(21) Appl. No.: 11/705,895

(22) Filed: Feb. 13, 2007

(51) Int. Cl. A23L 1/00; A23K 1/00; C12N 1/22; C12N 1/38

(52) U.S. Cl. 435/244; 426/2; 435/243; 435/252; 435/267

(57) ABSTRACT

A system including a harvested plant material degradation system, a microbial growth system in fluid contact with the harvested plant material degradation system, and an intermediary animal system in biomass-transfer interaction with the microbial growth system is disclosed. The intermediary animal can be fed to a product animal for consumption by the product animal. The intermediary animal can be worms, annelids, arthropods, mollusks, and/or fish. The intermediary animal can be provided for human consumption and/or consumption by a product animal, such as a crustacean, mollusk, fish, bird, pig, goat or cow. The product animal can be subsequently provided for human consumption. The harvested plant material degradation system can include polyculture plant material or photosynthetically produced material obtained from a single species of plant.

### BACKGROUND OF THE INVENTION

1. Field of the Invention

The present invention relates to a system for producing food and/or feed by generating a concentrated microbial biomass from the degradation of harvested plant material obtained from a monoculture or polyculture plant community, and providing the concentrated microbial biomass for consumption by an intermediary animal.

2. Description of the Related Art

Over the last 10,000 years, but most significantly in the last 100 years, a series of agricultural practices have been developed for the production of food for consumption by humans. These practices have culminated in today's modern agriculture in which a variety of plants and animals are grown and harvested for food. Modern agriculture usually includes the cultivation of land; selection, planting, and growing of selected single species of plants; irrigation of fields with groundwater from aquifers and surface waters; suppression of other plants that might compete with the selected plants by applying chemicals such as herbicides; suppression and control of various diseases and pests which attack the selected plants by applying chemicals; and the stimulation and promotion of growth and health of the selected plants by applying fertilizers to the fields.

Modern animal production practices usually include raising animals such as cattle, sheep, and goats for meat and/or milk by; grazing on rangelands including pastures, grasslands, and prairies which may be natural or may be planted or seeded with one or more of a variety of desirable plants, such as feed crops; or by feeding the animals grains and other plant products which are produced utilizing one or more of the previously listed modern agriculture practices.

While this modern agriculture has allowed for an unprecedented rise in the world's population, it has also resulted in serious environmental pollution and degradation. In the last 150 years, over half of the world's forests and wetlands have been destroyed so that the land could be used for grazing animals or cultivated for agricultural plant production. Much of the carbon which had been sequestered in the destroyed forests and wetlands has now been released to the atmosphere as carbon dioxide where many believe it contributes to global climate change and global warming.

The modern agricultural practices have also led to the pollution of ground and surface waters with nutrients, pesticides, and other chemicals. This reduces and threatens fish populations as well as drinking water supplies. Raising large numbers of animals through conventional techniques also releases significant quantities of greenhouse gases to the atmosphere where again many believe that these gases contribute to global climate change. Modern practices also require excessive irrigation, which both depletes aquifers and increases the salinization of soils.

The continual plowing and cultivation of the land and the widespread use of an increasing variety of pesticides has destroyed a large fraction of the topsoil that once existed. As the organic fraction of the soil has been oxidized it has also been exposed to erosion, which not only depletes the soil but also leads to additional pollution of the groundwaters, lakes, streams, rivers, and even the oceans. A further consequence of this pollution has been the reduction of desirable fish populations in the waters of the earth. Eutrification and decline in water quality, destruction of spawning and nursery habitat, and continual overfishing have depleted many of the populations of the most desirable fish used for human food.

Accordingly, the present invention has been developed in view of limitations, shortcomings and other disadvantages of conventional production practices.

### SUMMARY OF THE INVENTION

To resolve the environmental problems contributed to and created by modern agriculture this invention presents a novel method of producing food while sequestering large amounts of carbon compared to modern agriculture. It is an object of the present invention to provide a system including a harvested plant material degradation system, a microbial growth system in fluid contact with the harvested plant material degradation system, and an intermediary animal system in biomass-transfer interaction with the microbial growth system.

The harvested plant material degradation system can provide a substrate for the microbial growth system, and the harvested plant material degradation system can include polyculture plant material. The microbial growth system can produce a concentrated biomass, such as having a microbial concentration of at least of at least 10⁸ microbes per milliliter. The intermediary animal comprises worms, annelids, arthropods, mollusks, and/or fish. The system can further include a product animal such as a crustacean, mollusk, fish, bird, pig, goat or cow by the consumption of the intermediary animal.

It is another object of the present invention to provide a process including the steps of microbially degrading harvested polyculture plant material to form a concentrated microbial biomass, and providing the concentrated microbial biomass to an intermediary animal for consumption by the intermediary animal. The process can also include the step of harvesting the intermediary animal for use as a feed and/or food.

The harvested polyculture plant material can include photosynthetically produced material obtained from more than one species of plant, or photosynthetically produced material obtained from a single species of plant. The intermediary animal may include worms, annelids, arthropods, mollusks, and/or fish.

It is another object of the present invention to provide a food produced by microbially degrading harvested polyculture plant material to form a concentrated microbial biomass, and providing the concentrated microbial biomass to an intermediary animal for consumption by the intermediary animal.

It is yet another object of the present invention to provide a process for producing a product animal which includes the steps of providing a product animal growth area having an outlet for waste, providing a harvested plant material collection area having an outlet for degradation products, providing a microbial growth system for producing a bacterial biomass having an outlet for effluent, directing at least some waste from the outlet of the product animal growth area to the harvested plant material collection area, directing at least some degradation products from the harvested plant material collection area outlet to the microbial growth system, directing at least some of the microbial biomass produced in the microbial growth system to an intermediary animal for consumption by the intermediary animal, and directing the intermediary animal to product animal growth area for consumption by the product animal. The intermediary animal can include worms, annelids, arthropods, mollusks, and/or fish. The product animal can include crustaceans, mollusks, fish, birds, pigs, goats and/or cows.

It is a further object of the present invention to provide a process for producing a food including at least one food producing unit including a harvested plant material degradation system, a microbial growth system in fluid contact with the harvested plant material degradation system, and an intermediary animal system in biomass-transfer interaction with the microbial growth system. The process also includes at least one plant material production area, means for harvesting plant material from the plant production area for use as a substrate in the food producing unit, and means for delivering liquid effluents from the food producing unit to the plant production area.

The system of the present invention can also include growing a large and diversified plant community such as a forest, prairie, weed field or natural wetland, without using cultivation or pesticides, periodically harvesting a fraction of the plant material produced by the plant community, using this harvested plant material, or products made from this harvested plant material, as a biodegradable substrate which contains minimal toxic components and is not harmful to animals and/or humans, subjecting the plant material to a microbial bioconversion reaction in which some, or as much as possible, of the substrate is converted into a microbial biomass, feeding this microbial biomass as a sole, predominant, or partial food source to an intermediary animal, and providing the intermediary animal for consumption by a product animal or as a direct human food.

It is a further object of the present invention to provide an agricultural practice which does not use cultivation or pesticides and which sequesters significant amounts of carbon in a food production practice. The system of the present invention can be used to produce food from land which currently contains a wetland or is forested and which does not currently produce a significant source of food for human consumption. The system of the present invention can eliminate, or minimize pollution of ground and surface waters with nutrients, pesticides and other chemical compounds.

It is yet a further object of the present invention to provide food and/or feed that is produced in a manner which does not cause significant pollution to the general environment, and sequesters large quantities of carbon, thereby reducing the impact of atmospheric carbon dioxide on global warming. This invention may further enable feed and/or food to be produced on large land areas which are not currently producing feed and/or food, and to do this in an environmentally compatible fashion.

### BRIEF DESCRIPTION OF THE DRAWINGS

FIG. 1 is a schematic representation of a system in accordance with an embodiment of the present invention;

FIG. 2 is a schematic representation of a system including a harvested plant material degradation system, a microbial growth system, and an intermediary animal system combined with a processing system in accordance with an embodiment of the present invention;

FIG. 3 is a schematic representation of a system including a fish growing system in accordance with an embodiment of the present invention;

FIG. 4 is a schematic representation of a full scale production system in accordance with an embodiment of the present invention;

FIG. 5 is a schematic representation of a system including additional water retention and final effluent polishing features in accordance with an embodiment of the present invention;

FIG. 6 is a schematic representation of a system including waste treatment of a fish production system in accordance with an embodiment of the present invention;

FIG. 7 is a schematic representation of a system including a bioreactor, clarifier, and sand filter in accordance with an embodiment of the present invention;

FIG. 8 is a schematic representation of a system including high nutrient concentrations in the harvested plant material degradation system and microbial growth system in accordance with an embodiment of the present invention;

FIG. 9a is a schematic representation of a top view of a combined system including a harvested plant material degradation system, a microbial growth system, and an intermediary animal system in the same physically confined space in accordance with an embodiment of the present invention;

FIG. 9b is a schematic representation of a side view of the combined system shown in FIG. 9a;

FIG. 10a is a schematic representation of a top view of a system including a plant growing system and water treatment system in accordance with an embodiment of the present invention;

FIG. 10b is a schematic representation of a side view of the system shown in FIG. 10a;

FIG. 11a is a schematic representation of a top view of a system including a fish access zone in accordance with an embodiment of the present invention;

FIG. 11b is a schematic representation of a side view of the system shown in FIG. 11a;

FIG. 12a is a schematic representation of a top view of a system including additional waste treatment systems in accordance with an embodiment of the present invention;

FIG. 12b is a schematic representation of a side view of the system shown in FIG. 12a; and

FIG. 13 is a schematic representation of a system including food production units in accordance with an embodiment of the present invention.

### DETAILED DESCRIPTION OF THE INVENTION

The present invention includes a method of agricultural production in which diverse mixed plant communities are grown, maintained, and partially harvested in a periodic manner so that significant quantities of carbon are sequestered within the persisting plant communities. The periodically harvested plant material is collected and concentrated in a physically defined space. There it is microbially broken down, converted into a microbial biomass which is processed into food for human consumption or animal feeds, or is fed to one or more varieties of small intermediary animals which are in turn used directly for food, used as an animal feed, or used as a raw material for the production of processed foods and animal feeds. The animal excreta or unused byproducts of each stage of production may be recycled back to prior stages where they are used as inputs of nutrients and biodegradable raw materials. Water may also be recycled within the system and parts of the microbial growth systems and production lands can serve a water purification and filtration function.

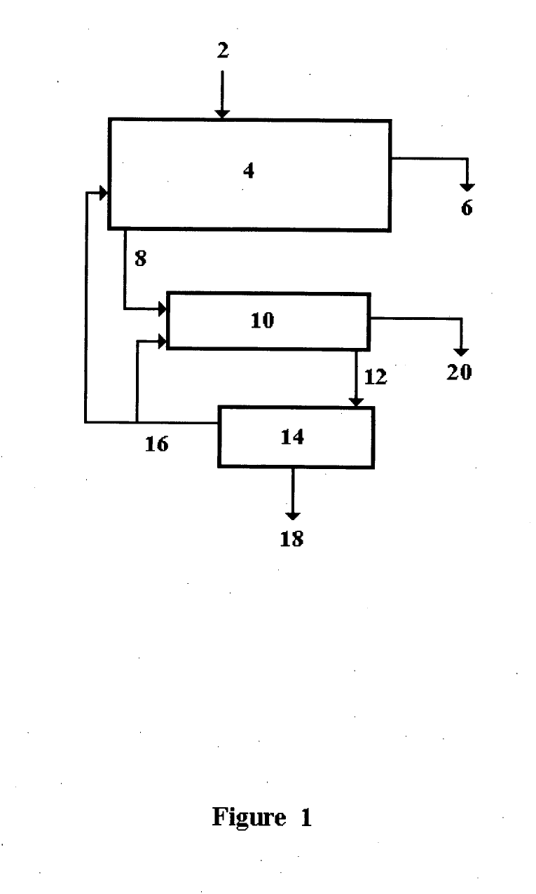

As shown in FIG. 1, harvested plant material 2 including material grown in terrestrial, wetland, or aquatic environments and byproducts produced from such material is loaded into a Harvested Plant Material Degradation (HPMD) System 4. As used herein, the term “harvested plant material” means material produced by a photosynthetic process which has been collected from the area where it was grown or concentrated in a part of the area where it was grown. The harvested plant material can be produced in a photosynthetic production system in which plant materials, including celluloses, lignins and/or starches, are produced via the process of photosynthesis on and/or in fields, grasslands, forests, wetlands, gardens, agricultural production lands, residential areas, suburban areas, and/or and aquatic systems. In one embodiment, the aquatic systems can include oceans, lakes, ponds, rivers, streams, and/or man made structures including lagoons, tanks, and containment structures. In one embodiment harvested plant material can include leaves, brush, trees, grass clippings, weeds, sawdust, organic food processing wastes, grasses, sedges, algae, aquatic plants, and/or agricultural residues. The plant communities from which the harvested plant material is obtained can comprise a polyculture of photosynthetic plants including a plurality of species of plants. The polyculture of plants can be a mixed and diverse community of plants, which grow in environments that are not plowed, mechanically cultivated, or contacted with pesticides and/or fertilizers. These can include plant communities growing in wetlands or aqueous environments. In another embodiment, harvested plant material can include plant material taken from monocultures or plants grown in cultivated environments. In another embodiment, harvested plant material can include newspaper, cardboard, sawdust, food wastes, or any substrate that is biodegradable, and either contains no toxic components or contains toxic components at a sufficiently low concentration so that the final use of material produced by the process of the invention is not considered toxic or harmful to animals and/or humans.

Plant material can be periodically harvested from an environment and this can be performed at a time and in a manner that minimizes the disruption of wildlife that uses that environment. Seeds, nutrients, and water may be introduced into an environment to enhance the amount and quality of plant biomass produced. By harvesting plant material that originates from a plant polyculture growing in an environment, the erosion of topsoil can be reduced and the nutrient and pesticide pollution associated with non point source agricultural runoff can be significantly limited. Land producing a polyculture of plant material can be more agriculturally productive since cultivation is not required, therefore the terrain does not need to be substantially flat.

As used herein, the term “Harvested Plant Material Degradation System” or “HPDM System” means a system for receiving harvested plant material and at least partially degrading at least a portion of the harvested plant material. Referring again to FIG. 1, the HPMD System 4 may include a tank, lined pond, or a solid floored pad, such as made of concrete or other hard impermeable material.

The harvested plant material may be contacted with an aqueous based liquid, such as submerged in fresh or salt water, have water sprayed or irrigated over and/or through the material, or subjected to an alternating cycle of submergence and irrigation. Nutrients, unused byproducts and animal excreta from subsequent production components, and/or microbial inoculations may be added to the harvested plant material to encourage microbial action, which breaks down the physical structure of the harvested plant material. This involves the breaking of the plant cell walls, the releasing of the cytoplasmic contents, and/or the complete or partial biodegradation of the cellulose, hemicellulose, and/or lignin, which constitute the structural material of the plants. This degradation may occur in any combination of aerobic, micro aerobic, or anaerobic environments, in air, or in fresh or salt water. Organisms involved in this process may include, but are not limited to, bacteria, protozoa, fungi, and algae. In one embodiment, non-biodegradable residues 6 can be periodically removed from the system and land applied. The HPDM System 4 can also include a tank, lined cell, silo, bunker silo, concrete pad and/or area of shaped land to collect rainfall and/or leachate.

Referring yet again to FIG. 1, small particulate and soluble degradation products from the HPMD System 4 can be introduced via fluid contacting means 8 into a Microbial Growth System 10. The Microbial Growth System 10 microbially degrades the small particulate and soluble degradation products to produce a microbial biomass. As used herein, the term “microbially degrading” means biologically converting organic biodegradable material or material produced by a photosynthetic process into microbial cells. As used herein, the term “microbial biomass” means an organic mass including at least one of bacteria, microorganisms, protozoa, fungi and/or algae. In one embodiment, the microbial biomass may exist as single cells and/or as multicellular aggregates. The microbial biomass can form floc, readily settleable aggregates, zoogleal and/or filamentous masses. In one embodiment, the microbial biomass may be a concentrated microbial biomass produced by settling, centrifugation and/or filtration. In another embodiment, the microbial biomass can have a concentration of at least 10⁸ microbial cells per ml. Fluid contacting means can include conventional piping, and the like, which allow the small particulate and/or soluble degradation products from the HPMD System 4 to contact microorganisms in the Microbial Growth System 10. As used herein, the term “Microbial Growth System” means a system or process in which microorganisms consume all or part of a material substrate and produce additional microorganisms and degradation byproducts from the substrate. The Microbial Growth System 10 may include any non-toxic substrate and may employ fixed film or suspended growth systems operating in aerobic, anaerobic, anoxic conditions, or any combination of these and which may involve the recycling of solids or liquids. The Microbial Growth System 10 may include standard wastewater treatment technologies such as Activated Sludge, Sequencing Batch Reactor, Trickling Filters, Rotating Biological Contactors, Aerobic or Anaerobic Digestion, or the like. It also may include a bioreactor or chemostat type system as used in the fermentation, pharmaceutical, or other biotechnology industries.

In one embodiment, the microbial growth system 10 utilizes microbial growth to produce a harvestable microbial biomass, which may incorporate the microbes, metabolic byproducts, residues or other non-degraded components of the HPMD System 4. The inputs to the Microbial Growth System 10 may be soluble or particulate in nature and may also include additional nutrients and/or unused byproducts from subsequent production components. This system may employ fixed film or suspended growth systems with or without recycling of solids or liquids. The microbes may grow in aerobic, anaerobic, or anoxic conditions in fresh or salt water, and may include, but not be limited to, facultative microbes, bacteria, protozoa, fungi, and unicellular or small algae. This system also includes a means of concentrating and harvesting the produced microbial biomass. This may include the formation and settling of a floc, gravity settling, filtration, centrifugation, or other means of solids separation.

In one embodiment, solid residues from the Microbial Growth System 10 are periodically collected by a variety of conventional dewatering technologies such as gravity settling, filtration, presses, centrifuges, or the like. Excess water is discharged from the Microbial Growth System 10 as a liquid effluent 20.

In one embodiment an HPMD System 4 can directly produce a microbial biomass and this can occur with or without a means of concentrating said microbial biomass. In another embodiment a microbial growth system can act directly on a substrate which has not been previously acted on by an HPMD System 4. Generally such an embodiment is used in cases where the substrate has low concentrations of cellulose, hemicellulose, or lignin, or is a readily biodegradable material such as a food, food byproduct, or animal waste or wastewater.

Referring again to FIG. 1, the microbial biomass collected and produced by the Microbial Growth System 10 is delivered via 12, usually by pumping or gravity flow, to an Intermediary Animal System 14. As used herein, the term “Intermediary Animal System” means a system in which at least one variety of species is grown or maintained, which feed upon a microbial biomass produced by a microbial growth system. Intermediary animals can include small fish, such as any fish fry or minnows, worms such as annelids (including Oligochaetes), mollusks, including clams, snails, oysters, and mussels, arthropods, including insects and larvae, and/or crustaceans, including shrimp, crabs, lobster, and crayfish.

Any external source of a microbial biomass could be used as the feed stream for the Intermediary Animal System 14 provided that sufficiently low concentrations of toxic constituents are present. Thus residues from wine or beer fermentations, sludges from conventional wastewater treatment plants, biomass residuals from manure management systems, etc. could be used as Intermediary Animal System 14 input sources.

In one embodiment, animal excreta from the intermediary animals can be recycled back to the Microbial Growth System 10 or the HPMD System 4. Animal excreta and/or unconsumed microbial biomass from the Intermediary Animal System 14 can be discharged along with any excess water as a subsystem effluent 16 which is recycled back to other production systems such as the HPMD System 4 or Microbial Growth System 10. Harvested intermediary animals 18 are periodically removed from the Intermediary Animal System 14. In one embodiment the intermediary animals are harvested by mechanically separating the intermediary animals from the system. In another embodiment, the intermediary animals are removed from the system by draining the tank or containment area, gravity settling, hooking, netting, filtering and/or other conventional mechanical separation procedures. In another embodiment, the intermediary animals may self harvest themselves by crawling, swimming, or otherwise moving themselves out of the Intermediary Animal System 14.

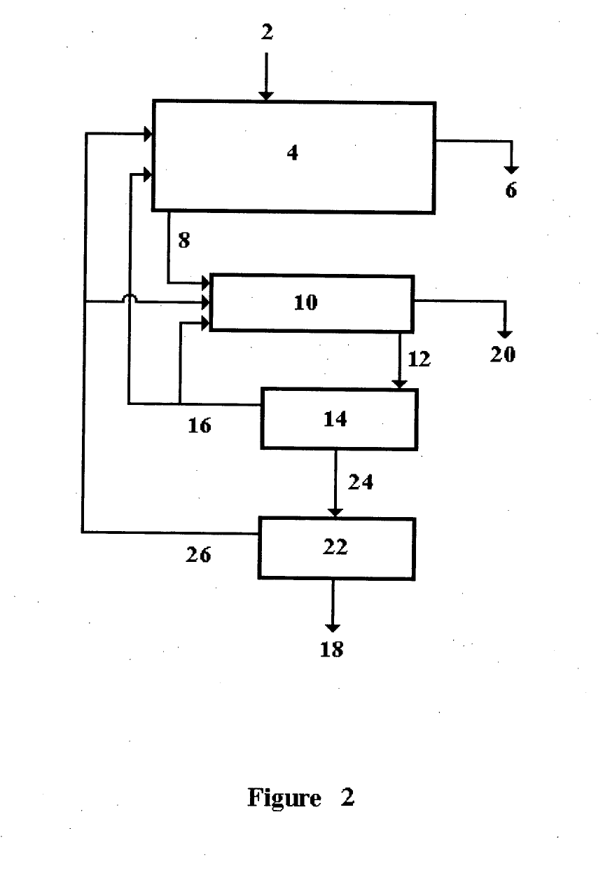

In another embodiment as shown in FIG. 2, the HPMD System 4, the Microbial Growth System 10 and the Intermediary Animal System 14 are combined with a Processing System 22. Here the harvested intermediary animals are transferred from the Intermediary Animal System 14 via 24 to the Processing System 22 where they are converted into animal feed, human food, or an edible substrate for processed foods 18 through conventional food processing techniques. In one embodiment, the intermediary animals can be processed into a fish food, such as a pelletized fish food, in the Processing System 22. Animal excreta, unused microbial biomass wastes from the Intermediary Animal System 14, and/or the unused byproducts from the Processing System 22 can be recycled via 26 to the Microbial Growth System 10 or the HPMD System 4 for reuse.

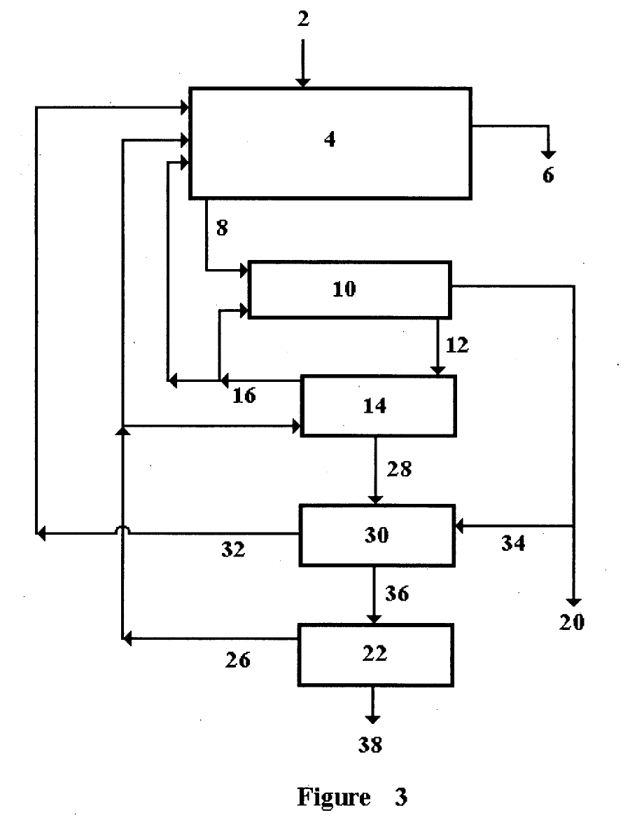

In another embodiment as shown in FIG. 3, harvested plant materials and nutrients 2 are introduced to the HPMD System 4 which directs degradation products via 8 to the Microbial Growth System 10, which produces a microbial biomass. The microbial biomass is sent via 12 to feed the Intermediary Animal System 14 which in turn produces intermediary animals, such as aquatic worms or oligochates, insect larvae, and/or crayfish. The intermediary animals are then provided via 28 to feed a product animal, such as a crustacean, mollusk, fish, pig, goat or cow, or other animal typically consumed by humans, in a Product Animal System 30. In one embodiment, the Product Animal System 30 is a fish growing system.

The fish growing system can produce fish that consume the animals grown in the Intermediary Animal System 14 as part or all of their diet. Feed other than the intermediary animal may be used to supplement the diet of the fish. In one embodiment, all or part of the effluent water from a tank housing product animals can be recycled back to the Microbial Growth System 10 or the HPDM System 4. In one embodiment, the recycled effluent can be taken from the bottom of a tank housing the fish in the fish growing system so that the solid excreta produced by the fish are removed. Clean water produced by the Microbial Growth System 10 can be used as influent water 34 for the Product Animal System 30 and excess water is discharged from the Microbial Growth System as effluent 20.

In one embodiment, fish can be sent via 36 to a Processing System 22 which produces cleaned fish and/or fish fillets 38. The unused byproducts from the Processing System 22 can be fed via 26 back to the Intermediary Animal System 14 and/or are recycled back to the HPMD System 4. Animal excreta, unused microbial biomass, and effluent from the Intermediary Animal System 14 are recycled via 16 back to the HPMD System 4 or the Microbial Growth System 10.

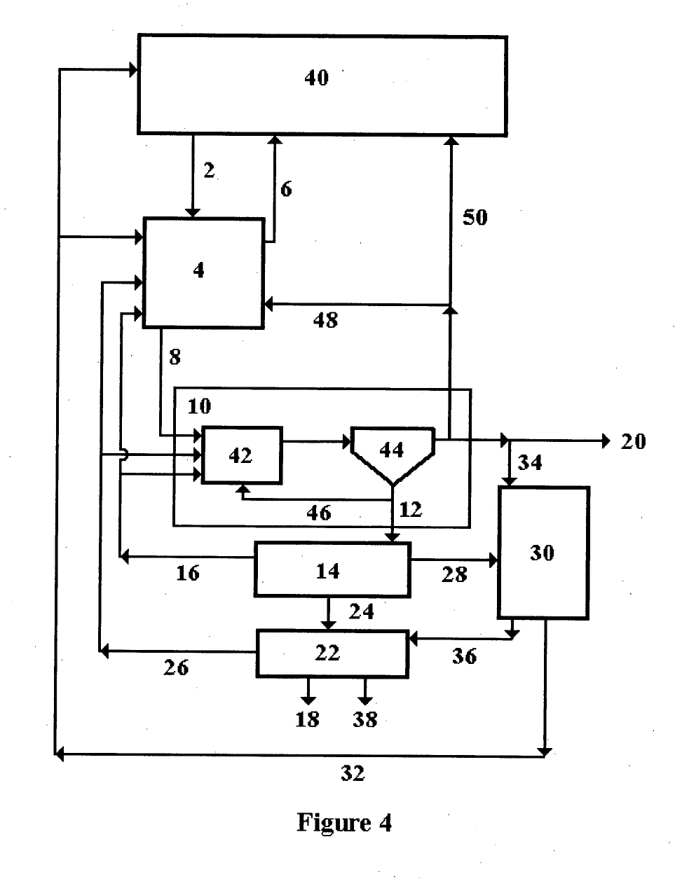

In yet another embodiment as shown in FIG. 4, a full scale production system of the invention includes an ecologically contained unit that operates on a given unit of land. As shown in FIG. 4, a Photosynthetic Production System 40 includes a forest, field, wetland, or body of water used to produce plant material. Harvested plant material can be collected at intermittent intervals, such as annually, from the Photosynthetic Production System 40. This harvested plant material will be transferred via 2 to an HPMD System 4. Nutrients can be added to optimize microbial growth. Non-biodegradable residues 6 may be periodically removed from the system and land applied to the Photosynthetic Production System 40.

As the harvested plant material is degraded the soluble and small particulate byproducts will be transferred via 8 to a Microbial Growth System 10 which may include an activated sludge type system including a bioreactor 42, clarifier 44, and recycle line 46. In one embodiment, the output from the HPMD System 4 may be further converted into microbial biomass which is embedded into a settleable floc structure. This settleable floc can then be substantially separated from the water in the clarifier 44 and the concentrated microbial biomass may be introduced via 12 into the Intermediary Animal System 14.

The effluent from the clarifier 44 may be used directly as a fresh water feed 34 to the Product Animal System 30, such as a fish raising tank if it has high enough water quality, or it may be diverted back via 48 to the HPDM System 4 or via 50 to the Photosynthetic Production System 40 for land application, or discharged as a final effluent 20.

In one embodiment, the concentrated microbial biomass, such as having about 2 percent solids, may be fed via 12 into large shallow trays or other structures in the Intermediary Animal System 14 where environments conducive to the rapid growth of a series of intermediary animals have been constructed. The intermediary animals, such as crayfish, aquatic worms, oligochaetes, clams, snails, scuds, insect larvae, minnows, and the like may eat the microbial biomass directly. In one embodiment, the intermediary animals can be frequently harvested to maintain optimal population densities for maximum consumption of the microbial biomass and production of the intermediary animals. Animal excreta and unused microbial biomass from the Intermediary Animal System 14 may be returned via 16 back into the HPMD System 4. The harvested intermediary animals may be fed via 28 to the animals in the Product Animal System 30, such as fish in a fish tank, or will be sent via 24 to a Processing System 22.

If the various systems so far described produce something other than a whole product animal, then a Processing System 22 may be included to convert the intermediary animals into a product that is typically consumed by humans. For example, fish, crayfish, clams, snails and the like may require cleaning and processing prior to sale for human consumption. In one embodiment, a Processing System 22 can include a fish cleaning and/or filleting operation. Here the final product would be the cleaned fish or the fish fillets. The unused byproducts produced of this processing operation may be returned back to the Microbial Growth System 10 or the Intermediary Animal System 30. In one embodiment, fish cleaning residues such as guts, heads, fins, bones, and the like can be fed to crayfish in the Intermediary Animal System 30. In a more elaborate form, the Processing System 22 may include a system for converting intermediary animals into a synthetic food. In another embodiment, the Processing System 22 may convert intermediary animals into a pelletized food for feeding to fish.

Referring again to FIG. 4, animal excreta, unused microbial biomass, and effluent from the Intermediary Animal System 30 can also be recycled via 26 back to the HPMD System 4 or the Microbial Growth System 10. Wastes and effluent from the Product Animal System 30 may be recycled via 32 back to the HPMD System 4 or the Photosynthetic Production System 40.

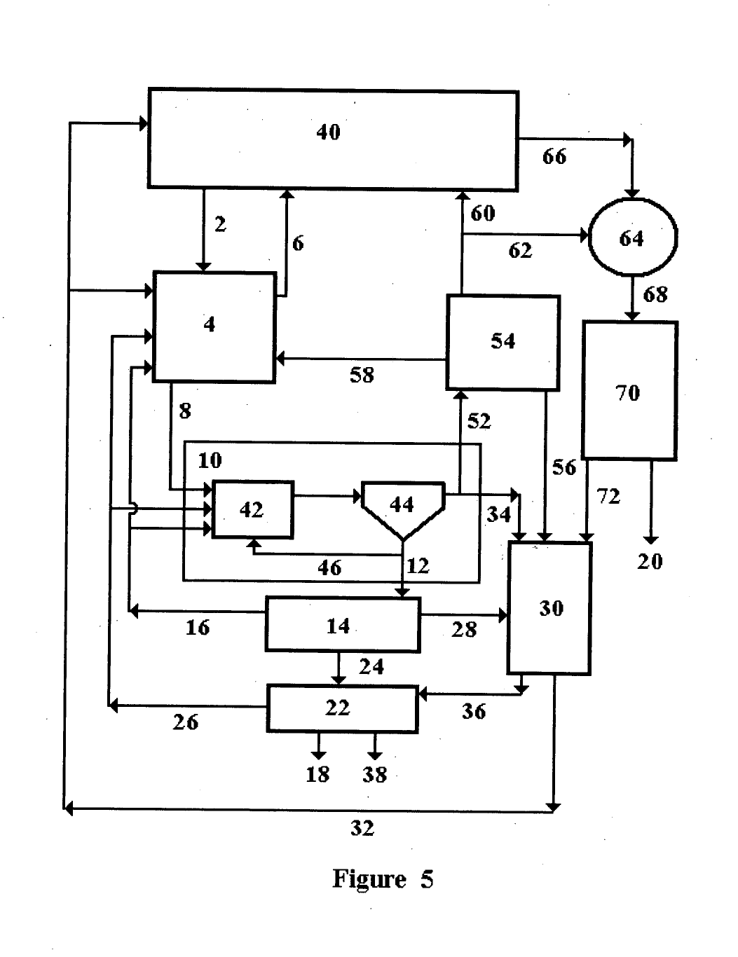

In yet another embodiment as shown in FIG. 5, a full scale production system of the present invention can include an ecologically contained unit that operates on a given unit of land with additional water retention and final effluent polishing features. In one embodiment, a Photosynthetic Production System 40 includes a forest, field, wetland, or body of water used to produce plant material. Harvested plant material may be collected intermittently or once a year from the Photosynthetic Production System 40. This harvested plant material may be transferred via 2 to an HPDM System 4. In one embodiment, nutrients may be added to optimize microbial growth. As the harvested plant material is degraded, the soluble and small particulate byproducts may be transferred via 8 to a Microbial Growth System 10, which may include an activated sludge type system including a bioreactor 42, a clarifier 44, and separated solids recycle line 46. Here the output from the HPMD System 4 may be further converted into microbial biomass in 42 in the form of a settleable floc structure. The settleable floc structure can then be separated from the water in the clarifier 44 and the concentrated microbial biomass may be recycled back to the bioreactor 42 via the separated solids recycle line 46, or introduced via 12 into the Intermediary Animal System 14.

The effluent from the clarifier 44 can be used directly as a fresh water feed via 34 to a Product Animal System 30, such as a fish raising tank if it has high enough water quality, or it may be diverted via 52 through a sand filter or a wetland 54 and then via 56 to the Product Animal System 30. In one embodiment, the effluent from the clarifier 44 is introduced via 52 into a constructed wetland for the purposes of aeration, additional filtration and removal of suspended solids, and nutrient removal. This polished effluent then may be used via 56 as the influent water for the Product Animal System 30 or may be recycled back to the HPDM System 4. The effluent may also be land applied via 60 back to the Photosynthetic Production System 40 or sent via 62 to a collection pond 64. From there it may be transferred via 68 for further treatment in a constructed polishing wetland 70. Effluent from the polishing wetland 70 can be used as a fresh water feed 72 to the Product Animal System 30, such as a fish raising tank. Excess effluent can be discharged from the polishing wetland 70 as a final effluent 20.

The concentrated microbial biomass, such as having about 2 percent solids, may be fed via 12 to the Intermediary Animal System 14 and distributed into large shallow trays or other structures where environments conducive to the rapid growth of a series of intermediary animals have been constructed. The intermediary animals, such as crayfish, aquatic worms, oligochaetes, clams, snails, scuds, insect larvae, minnows, and the like may eat the microbial biomass directly. The intermediary animals may be frequently harvested to maintain optimal population densities for maximum consumption of the microbial biomass and production of the intermediary animals. Animal excreta and unused microbial biomass from the Intermediary Animal System 14 may be returned via 16 back into the HPMD System 4. The harvested intermediary animals may be fed via 28 to the animals in the Product Animal System 30, such as fish in a fish raising tank, or sent via 24 to a Processing System 22 which produces feed, food or processed food 18 from the intermediary animals.

In one embodiment, fish are sent via 36 to a Processing System 22 which produces cleaned fish and fish fillets 38. The unused byproducts from the Processing System 22 are recycled back via 26 to the HPMD System 4 or the Microbial Growth System 10. Animal excreta, unused microbial biomass, and effluent from the Intermediary Animal System 14 and the Product Animal System 22 are recycled back to the HPMD System 4 or the Photosynthetic Production System 40, as described earlier.

In another embodiment effluent from a fish raising tank may be returned via 32 to the HPMD System 4. In another embodiment, the effluent may also be diverted to the Photosynthetic Production System 40 depending on flow and water quality requirements for the desired fish to be raised. Runoff from the Photosynthetic Production System 40 may be collected via 66 in a holding area 64, such as a pond, and then transferred via 68 for further treatment in a constructed polishing wetland 70. Effluent from the polishing wetland 70 can be used as a fresh water feed 72 to the Product Animal System 30, such as a fish raising tank. Excess effluent can be discharged from the polishing wetland 70 as a final effluent 20.

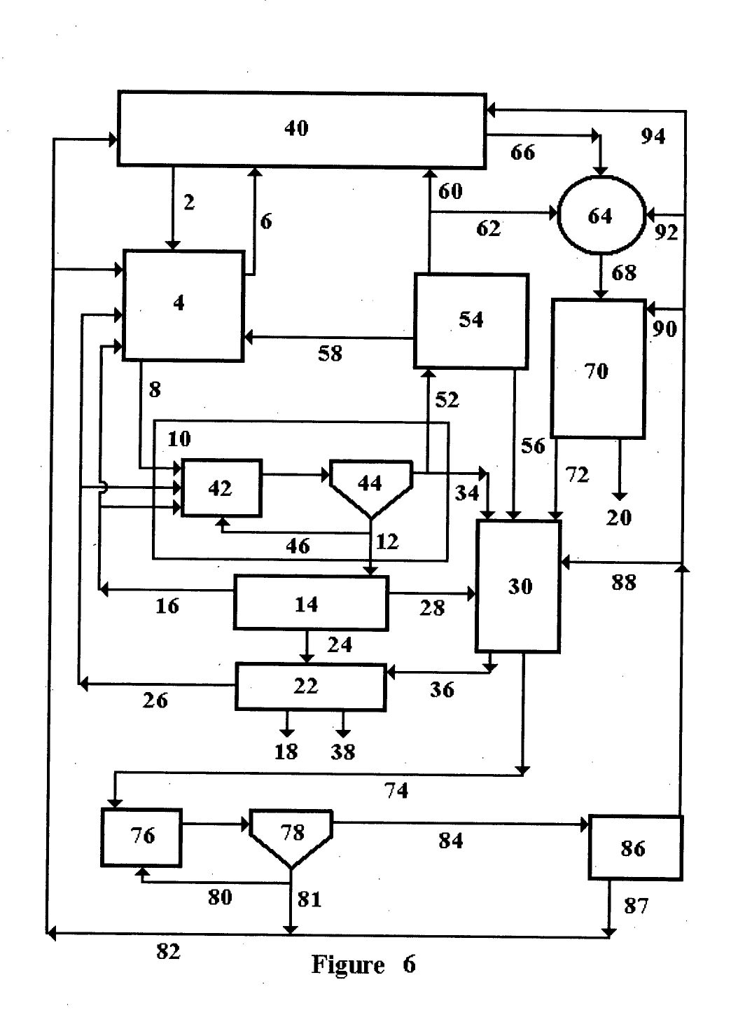

A further expansion on this system with additional water retention and treatment provisions is shown in FIG. 6. In this embodiment, the system described in FIG. 5 is expanded by including additional wastewater treatment of the Product Animal System 30, which in FIG. 6 shall be referred to with reference to a Fish System 30a. As shown in FIG. 6, the Fish System 30a effluent is transferred via 74 for further treatment with an activated sludge type water treatment system including a bioreactor 76 and clarifier 78 with a concentrated solids recycle loop 80. The Fish System 30a effluent can be microbially treated in the bioreactor 76 and then transferred via 77 to the clarifier 78 where the microbial solids are partially separated from the liquid. Clarifier 78 effluent may be transferred via 84 to a sand filter 86 for further effluent polishing. Waste solids 81 from the clarifier 78 and backwash 87 from the sand filter 86 can be recycled via 82 to the Plant Production System 40 or the HPMD System 4. The clean water effluent from the sand filter 86 can be recycled via 88 to the Fish System 30a, recycled via 92 to a collection pond 64, recycled via 90 to the polishing wetland 70, or recycled via 94 back to the Plant Production System 40 for land application. Excess water is discharged via 20 as final effluent from the polishing wetland 70.

In the various embodiments of the invention described herein, the HPMD System 4 and Microbial Growth System 10 may operate with total nitrogen concentrations, and in particular with ammonia and ammonium ion concentrations, which are sufficiently low such that the resulting concentrations of ammonia and ammonium ions in the Product Animal System 30 or the Fish System 30a do not interfere with the growth and health of the fish or other product animal. In an alternative embodiment the influent to and effluent from the Product Animal System 30 or the Fish System 30a is uncoupled from the HPMD System 4 and the Microbial Growth System 10, and an additional water treatment system is installed to maintain water quality in the Animal System 30 or the Fish System 30a.

The addition of the additional treatment system for the fish/animal wastes allows for much higher concentrations of nutrients, particularly various forms of nitrogen, to be maintained in the HPMD System 4 and the Microbial Growth System 10. This in turn may increase the rate of microbial conversion of harvested plant material to microbial biomass from that attainable in low nitrogen concentration systems.

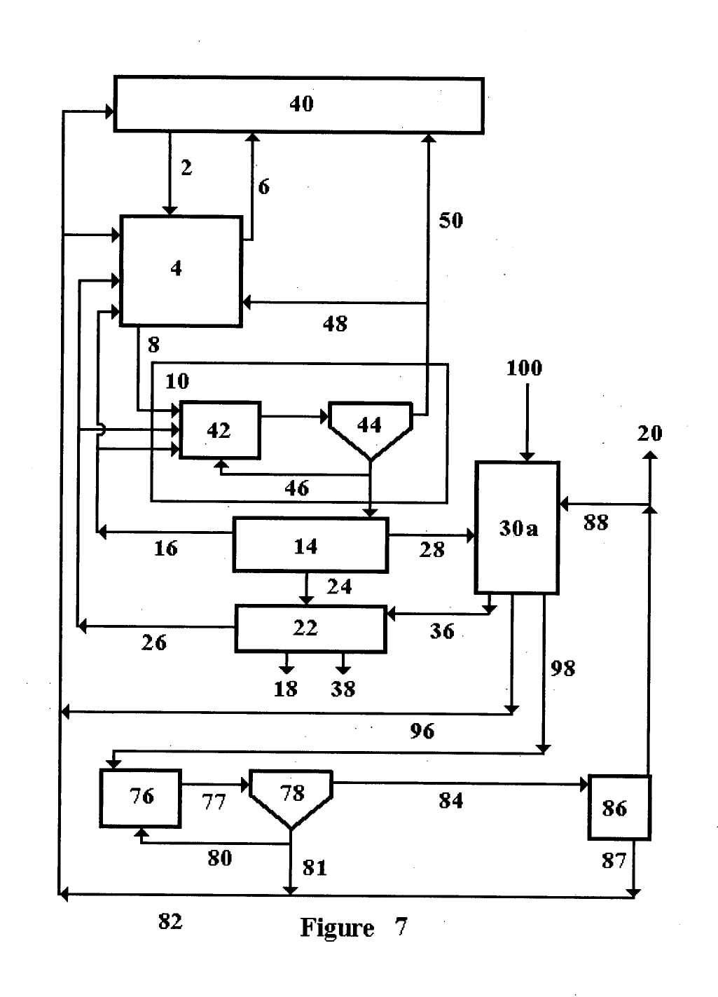

As shown in FIG. 7 the additional treatment system added to the system shown in FIG. 4 includes an activated sludge type system including a bioreactor 76 and a clarifier 78 followed by a sand filter 86. In this embodiment, fish wastes from the bottom of the Fish System 30a may be transferred via 96 to the Plant Production System 40 or the HPMD System 4. Effluent from the Fish System 30a may be transferred via 98 to the bioreactor 76 where soluble and small particulate fish wastes are microbially degraded and converted into biomass. The biomass may then be sent via 77 to the clarifier 78 where it is separated from the majority of the water stream. The collected solids are sent via 81 and/or 82 to the Plant Production System 40 or the HPMD System 4. The clarified water is sent via 84 for further treatment in the sand filter 86. Clean water from the sand filter 86 may be sent via 88 to the Fish System 30a or discharged 20. Backwash wastes from the sand filter 86 can be sent via 87 and/or 82 to the Plant Production System 40 or the HPMD System 4. Similar wastewater treatment systems such as trickling filters, rotating biological contractors, various biological nutrient removal systems, suspended growth systems, fixed film systems and the like may be used in as partial or complete replacements for the above-described activated sludge type system. In this embodiment some clean external water 100 may be periodically added to the Fish System 30a to maintain overall water balance.

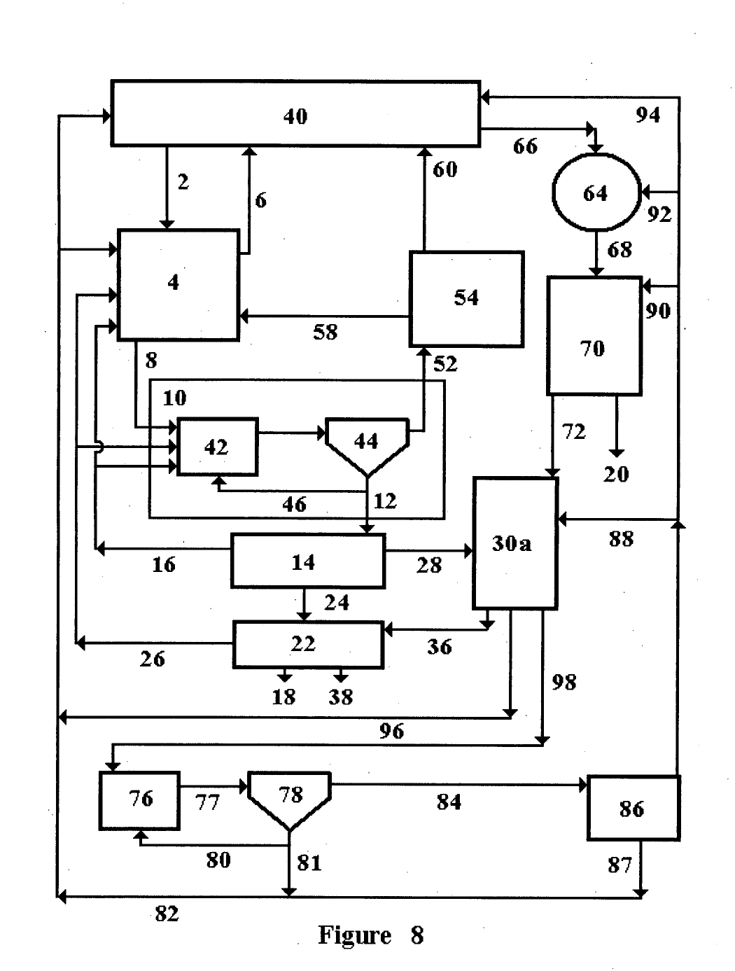

In another embodiment shown in FIG. 8, the configuration allowing for high nutrient concentrations for the HPMD System 4 and Microbial Growth System 10, as shown in FIG. 7, is integrated with further wetland water polishing features for reuse in the Fish System 30a or Plant Production System 40, or for final discharge 20. In this embodiment the sand filter 86 effluent may be directed back to the Plant Production System 40 via 94 or may be directed to a collection pond 64 via 92, or a polishing wetland 70 via 90, or to the Fish System 30a via 88. The pond and polishing wetland can provide for additional water sequestering capability as well as for further improvement of water quality.

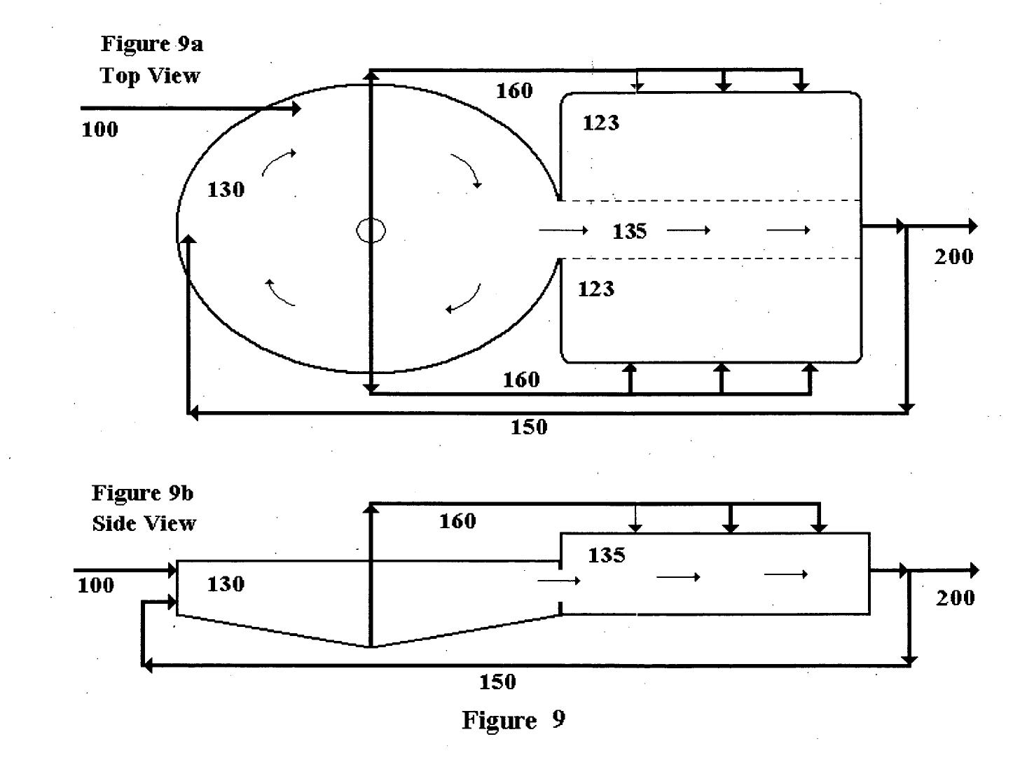

In another embodiment of the invention shown in FIGS. 9a and 9b, the HPMD System, Microbial Growth System, and the Intermediary Animal System described in earlier figures are each located in the same physical space to form a Combined System 123. In the Combined System, the HPMD System, the Microbial Growth System, the Intermediary Animal System, and the Product Animal System or Fish System are all combined in one contained volume such as a tank or pond with defined influent and effluent streams. In this embodiment the HPMD System may be confined to a part of the Fish Growing system, called the HPMD zone. The Microbial Growth System and the Intermediary Animal System may also be physically contained within, or will predominately reside within, the HPMD zone. The interface between the HPMD zone and the rest of the Product Animal System or Fish System will be such that water can freely flow between the two systems and that small fish can freely enter all or part of the HPMD zone to feed on microbes and intermediary animals which grow and reside within the HPMD zone. In one embodiment, larger product animals, such as fish, may be unable to enter the HPMD zone but will be able to feed on small fish and other intermediary animals which will grow within the HPMD and migrate out of this zone into the rest of the Product Animal System or Fish System.

Waste materials which collect at the bottom of the Fish System, particularly including fish excreta, can be collected and pumped as a recycle back to the HPMD zone where it can serve as a source of nutrients for the microbial degradation processes. In this embodiment the influent stream will enter the Fish System at some distance from the HPMD zone and the effluent stream will exit the total system through the HPMD zone.

As shown in FIGS. 9a and 9b, the Combined System 123 can be directly connected to a Fish System 130 in a manner so that fish can directly access and feed on the Intermediary Animals and microbial biomass produced in the Combined System 123.

Referring again to FIGS. 9a and 9b, the Fish System 130 may include a circular shallow fish tank or lined pond having a slightly sloped conical bottom. This may be directly connected to a tank or lined pond which would contain the Combined System 123. An influent water stream 100 may enter the fish tank at an angle inducing a slight circular movement of the water in the fish tank. An effluent stream 200 would leave the Combined System 123 area, preferably at a location furthest from the Fish System 130. Part of this effluent stream would be recycled via stream 150 back to the fish tank where it could be reintroduced at an angle so as to enhance the circular movement of the water in the fish tank.

In the Combined System 123, harvested plant material such as brush, branches, plant stalks, leaves, wood chips, and the like may be placed in an arrangement such that water and/or intermediary animals can penetrate the harvested plant material structures, i.e., to move in between branches. In one embodiment, aeration may be applied to the harvested plant material, such as at the bottom of the area where the harvested plant material is introduced such that the water surrounding the harvested plant material has a measurable dissolved oxygen level. Microbes may grow on the surfaces of the harvested plant material structures, thereby degrading the harvested plant material and creating new microbial biomass. Intermediary animals may then access and feed on this microbial biomass throughout the Combined System 123.

Generally, the harvested plant material in the Combined System 123 is submerged under water at all times. However, in some embodiments of the present invention, a portion, or even all, of the harvested plant material may be stacked above the water level. In these embodiments the harvested plant material may be periodically or continuously irrigated with water and/or nutrients to promote the growth of the microbial biomass.

Referring again to FIGS. 9a and 9b, the slight circular movement of the water in the fish tank of the Fish System 130 induced by the appropriate location and direction of the influent stream 100 and the system recycle flow 150 may allow fish excreta to be collected at the bottom or apex of the fish tank in the Fish System 130. From there it may be pumped via stream 160 back to the Combined System Area 123 where the fish excreta may be filtered out of the water by the harvested plant material. In the embodiments in which some or all of the harvested plant material is stacked above the water level, some or all of the recycled flow containing fish excreta and/or added nutrients are sprayed on to the top of the harvested plant material. This may be accomplished on a continuous or periodic basis such that the harvested plant material remains substantially wet to facilitate microbial growth. In these embodiments, blowers may be used instead of submerged aerators to supply adequate oxygen for the biological degradation of the harvested plant material.

The recycled wastes from the Fish System 130 may provide nutrients for the microbes degrading the harvested plant material. Additional nutrients could be added to this recycle stream to enhance the microbial degradation of the harvested plant material. In one embodiment, a fish access zone 135 can be provided to allow fish to enter the Combined System Area 123 to feed on the microbial biomass and the intermediary animals. Thus, the connection of the Fish System 130 to the Combined System Area 123 may be large enough to allow even large fish to easily pass between the two systems. By allowing this access, fish may feed on both the bacterial biomass and intermediary animals which live and grow in the Combined System Area 123.

In one embodiment, the system shown in FIGS. 9a and 9b can be constructed such that it is uncovered and open to the atmosphere, or covered with a greenhouse type structure or other material covering. Having this covering could retain heat within the system, which in cold climates could prevent freezing and could help maintain appropriate temperatures to promote the growth of microbes, intermediary animals, and product animals, such as fish. The covering could also prevent rainfall from entering the system or could reduce water loss from the system through evaporation.

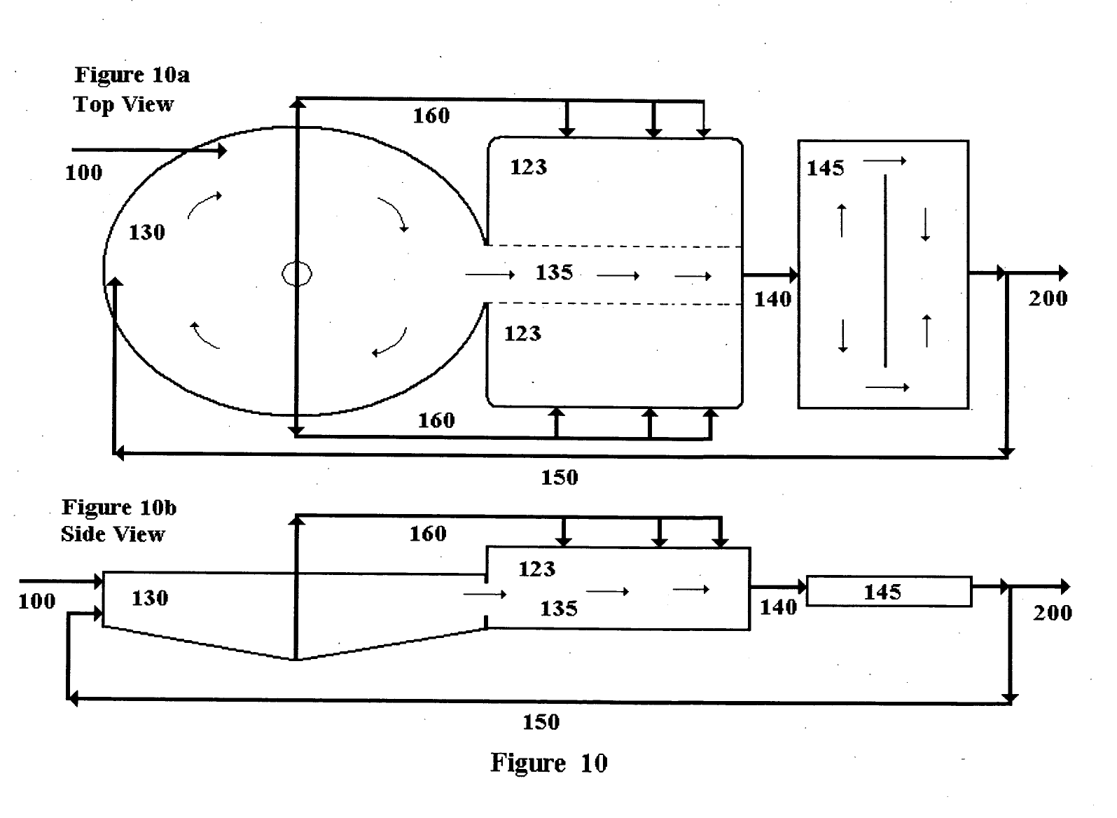

In another embodiment of the present invention, the system shown in FIGS. 9a and 9b may be connected to a plant growing system or a water treatment system. This embodiment is shown in FIGS. 10a and 10b in which the effluent from the Combined System 123 is transferred via 140 to a Plant Growing System 145 including a shallow tank, lined pond, or natural pond where plants are grown in the water. These plants may include food plants such as watercress for cold climates or water chestnuts for warm climates, or they could comprise wetland plants adapted to the climate of the system. The plants could have a cleaning effect of the effluent stream, removing nutrients which the plants could use for growth, and filtering out particulate material. The Plant Growing System 145 could be uncovered or it could be part of a greenhouse structure which allows sunlight to enter for plant growth, or it could be covered by other structures which have artificial light to enable plant growth.

As shown in FIGS. 10a and 10b, structures may be placed in the Plant Growing System 145 to establish a long flow path which would produce a cleaner effluent, such as having a lower nutrient and/or particulate content, which could then be discharged 200 or recycled 150 back to the Fish System 130. The Plant Growing System 145 could contain several parallel but separate channels or flow paths such that one channel could be taken out of service for harvesting of plants, or cleaning and maintenance of the structure, without impairing the filtration and cleaning function from treating the effluent with alternative channels.

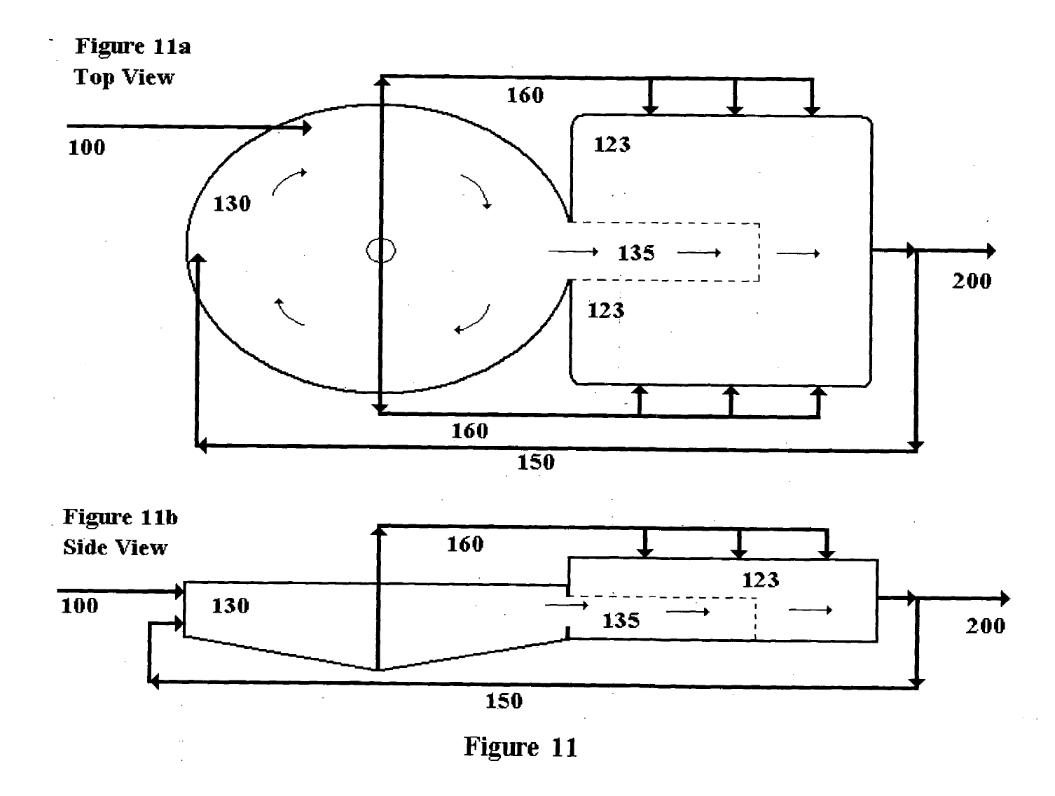

As shown in FIGS. 11a and 11b, the system shown in FIGS. 10a and 10b can be modified such that the effluent from the Fish System 130 passes through the Fish Access Zone 135 and is then directed through at least a part of the Combined System 123 before emerging as effluent for recycle 150 or discharge 200.

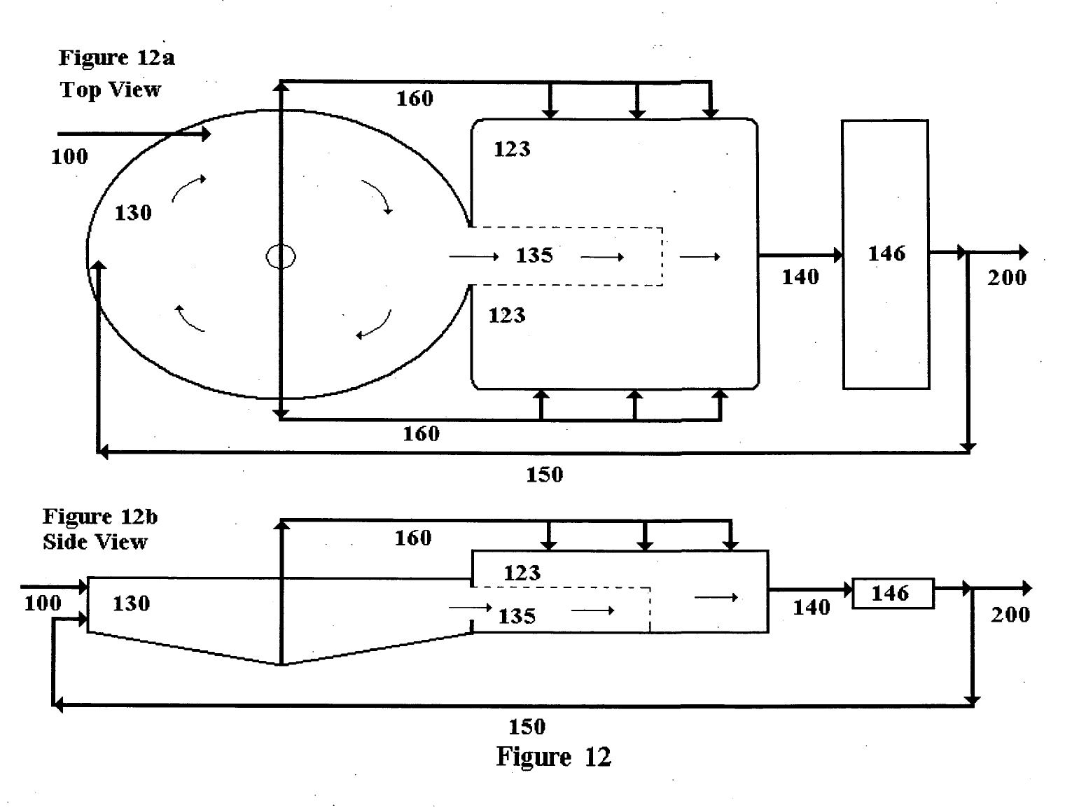

In another embodiment of the invention as shown in FIGS. 12a and 12b, the system shown in FIGS. 11a and 11b is connected via 140 to a further water treatment system 146 utilizing one or more of a variety of conventional wastewater treatment technologies. This may include, without limitation, activated sludge systems, trickling filter systems, rotating biological contactors, or other types of conventionally known treatment systems. It should be noted that the water treatment systems 145 and 146 may be interchangeably connected to the systems described in FIGS. 9a and 9b and 11a and 11b, and these systems could be covered or uncovered as a function of local conditions including climatic conditions.

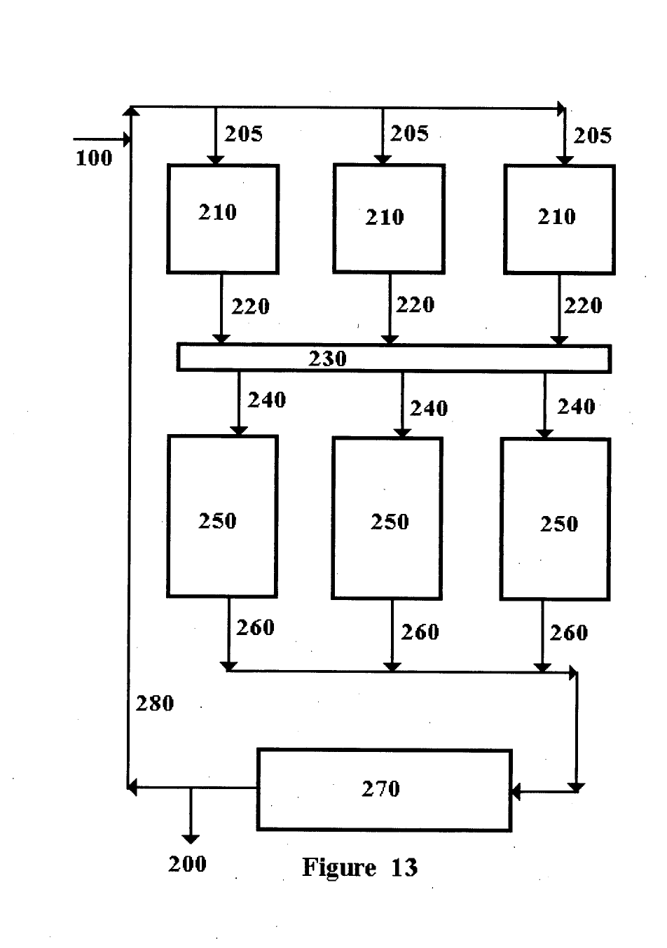

The systems of the present invention can also be utilized to create a fundamentally new method of producing food from natural environments. A preferred embodiment of this feature of the invention is shown in FIG. 13. Here a food, feed, or product animal, such as fish, is produced in one or more of a series of Food Production Units 210 which may include without limitation any of the systems previously described herein. These Food Production Units 210 can be located within a natural environment such as a forest, prairie, wetland, or the like and can use harvested plant material collected in an ecologically sustainable manner from these natural environments. This might include partial and periodic harvesting of plants and plant material at various times of year that would minimize or eliminate negative effects on wildlife inhabiting those environments.

The liquid effluents from the Food Production Units 210 may be transferred via 220 to a manifold, pipe, channel, canal or stream 230 and conveyed via 240 to a series of Plant Production Units 250 which could comprise flooded or irrigated fields, paddys, production wetlands or the like and which may be lined or unlined depending on soil conditions, water table elevation or other local conditions. These Plant Production Units 250 may be used for growing wild rice or watercress in cold climates or rice and water chestnuts in warm climates. Alternatively, any cultivated plant for which continuous or periodic flood irrigation is applicable could be used.

The effluents from the Plant Production Units 250 are collected via 260 in a final water polishing and distribution system 270. This unit may comprise a wetland, channel or canal that contains plants for filtration of particulate matter and removal of nutrients. The effluent from this final unit may be recycled via 280 and distributed via 205 to the Food Production Units 210, or may be discharged via 200 for use for irrigation in the containing natural environment or discharged to various bodies of water such as wetlands, ponds, streams, or the like within the containing natural environment.

All of the components of this system, except sometimes for the Plant Raising Areas 250, can be located within the natural environment that produces the harvested plant material used by the system. Thus the Food Production Units 210 and the various wetlands, channels, canals and the like represented by 230 and 270 could reside underneath a forest or savannah canopy or the weeds, grasses, bushes and shrubs of various wetland or grassland environments. This would allow significant food and feed production from land currently not used for agricultural production of food and feed and could do so in a manner that would sequester much greater amounts of carbon and water than is possible with conventional agricultural land which is used for the cultivation of grain and vegetable crops.

The above mentioned component systems can be connected via a variety of forward flows whereby the products of the component are transferred to the next system component, and by a series of recycle flows whereby the component byproducts and animal excreta are recycled back to prior components for reutilization within the production process.

In one embodiment the recycle flows originates with the Intermediary Animal System, the Processing System, the Fish System, or the Aeration Wetland, and is directed to the Microbial Growth System, the Harvested Plant Material Degradation System, or the Photosynthetic Production System. The selection of destinations and the partitioning of flows if more than one destination is chosen can be a major part of the management and control system for the total process.

A system of mass balance accounting is also used to control and manage the production process. This mass balance approach will track some or all of the following chemical elements; carbon, hydrogen, oxygen, nitrogen, phosphorus, sulfur, sodium, potassium chloride, calcium, magnesium, iron, manganese, copper, zinc, and nickel. In general carbon dioxide and water will be fixed into plant material (carbohydrate, cellulose, etc.) in the Photosynthetic Production System. Minerals, salts, and nutrients will be extracted from the earth in the fields and wetlands, and a series of products including fish, feeds, and processed foods will be removed from the system. To balance the elements removed in the products nutrients and minerals will need to be added to maintain the chemical balance in the production lands and in the system itself.

Because parts of the system are open to the surrounding environment it is necessary to maintain a water balance throughout the system. Rain, snow and other forms of precipitation will enter the system and evaporation and evapotranspiration will remove water from the system. Any imbalance in the water inventory will be compensated for by either adding water from an external source or discharging excess water to the environment. If excess water must be discharged it usually will come from the effluent flow from the Microbial Growth System which will pass through an aeration wetland or a filter (such as a sand filter). The excess water will then be further treated by land applying it to the Production Fields where it may overflow into a Collection Pond or by discharging it directly to a Collection Pond or a treatment wetland. Once there it will normally flow through a final polishing wetland and then be discharged to the environment. Other forms of water treatment technology may be applied to meet mandated discharge water quality criteria.

Whereas particular embodiments of this invention have been described above for purposes of illustration, it will be evident to those skilled in the art that numerous variations of the details of the present invention may be made without departing from the invention as defined in the appended claims.

What is claimed is:

1. A system, comprising:
a harvested plant material degradation system;
a microbial growth system in fluid contact with the harvested plant material degradation system; and
an intermediary animal system in biomass-transfer interaction with the microbial growth system.

2. The system of claim 1, wherein the harvested plant material degradation system is a substrate for the microbial growth system.

3. The system of claim 1, wherein the harvested plant material degradation system comprises polyculture plant material.

4. The system of claim 3, wherein the polyculture plant material comprises cellulosic material, brush, grasses, tree branches, tree trunks, leaves, food starch, paper, lumber, sawdust, plant fibers, fruit or vegetable products or byproducts, succulent plants, and/or seeds.

5. The system of claim 3, wherein the polyculture plant material is contacted with an aqueous-based liquid by submerging the polyculture plant material within the aqueous-based liquid, spraying the aqueous-based liquid on the polyculture plant material, and/or irrigating the polyculture plant material.

6. The system of claim 3, wherein the polyculture plant material is contacted with a microbial inoculation and/or nutrients.

7. The system of claim 1, wherein the microbial growth system comprises a fixed film and/or suspended growth system.

8. The system of claim 1, wherein the microbial growth system promotes growth of microbes in aerobic, anaerobic, and/or anoxic environments.

9. The system of claim 1, wherein the microbial growth system produces a concentrated biomass.

10. The system of claim 9, wherein the concentrated biomass has a concentration of at least 10⁸ microbes per milliliter.

11. The system of claim 9, wherein the concentrated biomass can be concentrated by gravity settling, filtration, and/or centrifugation.

12. The system of claim 1, wherein the intermediary animal system comprises intermediary animals for consuming a concentrated biomass produced in the microbial growth system.

13. The system of claim 12, wherein the intermediary animal comprises worms, annelids, arthropods, mollusks, and/or fish.

14. The system of claim 1, further comprising a harvesting system for collecting intermediary animals from the intermediary animal system after the intermediary animals have consumed at least a portion of a concentrated biomass produced in the microbial growth system.

15. The system of claim 1, further comprising a product animal, wherein intermediary animals produced in the intermediary animal system are provided to the product animal for consumption by the product animal.

16. The system of claim 15, wherein the product animal is a crustacean, mollusk, fish, bird, pig, goat or cow.

17. A process, comprising the steps of:
microbially degrading harvested polyculture plant material to form a concentrated microbial biomass; and
providing the concentrated microbial biomass to an intermediary animal for consumption by the intermediary animal.

18. The process of claim 17, further comprising the step of harvesting the intermediary animal for use as a feed and/or food.

19. The process of claim 17, further comprising contacting the harvested polyculture plant material with a microbial inoculation and/or nutrients.

20. The process of claim 17, wherein the microbial inoculation and/or nutrients comprises organisms, organism excreta, microbial biomass and/or byproducts of the microbial biomass, intermediary animals or byproducts of the intermediary animals, and/or inorganic or organic fertilizers or other nutrient containing materials.

21. The process of claim 17, wherein the harvested polyculture plant material comprises photosynthetically produced material obtained from more than one species of plant.

22. The process of claim 17, wherein the harvested polyculture plant material comprises photosynthetically produced material obtained from a single species of plant.

23. The process of claim 17, wherein the intermediary animal comprises worms, annelids, arthropods, mollusks, and/or fish.

24. The process of claim 17, wherein the step of microbially degrading occurs in a harvested plant material degradation system.

25. The process of claim 24, wherein the harvested plant material degradation system comprises a tank, lined cell, silo, bunker silo, concrete pad and/or area of shaped land to collect rainfall and/or leachate.

26. The process of claim 17, wherein the microbial biomass is formed in a microbial growth system comprising an activated sludge system, sequencing batch reactor, suspended growth system, fixed film growth system, rotating biological contactor, trickling filter, bioreactor, chemostat, and/or other microbial growth system.

27. A food produced by the method of:
microbially degrading harvested polyculture plant material to form a concentrated microbial biomass; and
providing the concentrated microbial biomass to an intermediary animal for consumption by the intermediary animal.

28. The food of claim 27, further comprising the step of harvesting the intermediary animal and providing the intermediary animal to a product animal for consumption by the product animal.

29. The food of claim 28, wherein the product animal is a crustacean, mollusk, fish, bird, pig, goat or cow.

30. The food of claim 27, wherein the intermediary animal is a worm, annelid, arthropod, mollusk, or fish.

31. A process for producing a product animal, comprising:
providing a product animal growth area having an outlet for waste;
providing a harvested plant material collection area having an outlet for degradation products;
providing a microbial growth system for producing a bacterial biomass having an outlet for effluent;
directing at least some waste from the outlet of the product animal growth area to the harvested plant material collection area;
directing at least some degradation products from the harvested plant material collection area outlet to the microbial growth system;
directing at least some of the microbial biomass produced in the microbial growth system to an intermediary animal for consumption by the intermediary animal; and
directing the intermediary animal to product animal growth area for consumption by the product animal.

32. The process of claim 31, wherein the product animal is a crustacean, mollusk, fish, bird, pig, goat or cow.

33. The process of claim 31, wherein the intermediary animal comprises worms, annelids, arthropods, mollusks, and/or fish.

34. The process of claim 31, wherein the harvested plant material comprises photosynthetically produced material obtained from more than one species of plant.

35. The process of claim 31, wherein the harvested plant material comprises photosynthetically produced material obtained from a single species of plant or a monoculture.

36. The process of claim 31, wherein the harvested plant material collection area, the microbial growth system, and the intermediary animals are all positioned at least in part within a single combined system structure.

37. The process of claim 36, in which the single combined system further comprises a fish holding area structured to allow fish to enter the combined system and consume at least a part of the microbial biomass and/or the intermediary animals.

38. The process of claim 31, wherein the intermediary animal comprise worms, annelids, arthropods, mollusks, and/or fish.

39. The process of claim 31, further comprising adding a microbial inoculation and/or nutrients to the harvested plant material to assist in microbial degradation.

40. The process of claim 39, wherein the microbial inoculation and/or nutrients comprises organisms, organism excreta, microbial biomass and/or byproducts of the microbial biomass, intermediary animals and/or byproducts of the intermediary animals, and/or inorganic or organic fertilizers.

41. A process for producing a food, comprising:
at least one food producing unit comprising:
a harvested plant material degradation system,
a microbial growth system in microbial communication with the harvested plant material degradation system; and
an intermediary animal system in biomass-transfer interaction with the microbial growth system.
at least one plant material production area;
means for harvesting plant material from the plant production area for use as a substrate in the food producing unit; and
means for delivering liquid effluents from the food producing unit to the plant production area.

42. The process of claim 41, wherein the harvested plant material degradation system is a substrate for the microbial growth system.

43. The process of claim 41, wherein the harvested plant material degradation system comprises polyculture plant material.

44. The process of claim 41, further comprising means for delivering liquid effluents from the plant production area to the food producing unit.

45. The process of claim 41, wherein the food comprises worms, annelids, arthropods, mollusks, and/or fish.
